(19) United States
(12) Patent Application Publication
(10) Pub. No.: US 2017/0348140 A1
(43) Pub. Date: Dec. 7, 2017

(54) COLOSTOMY POUCH SUPPORT BAG

(71) Applicant: Cheryl Jean Riedel, Hermosa Beach, CA (US)

(72) Inventor: Cheryl Jean Riedel, Hermosa Beach, CA (US)

(21) Appl. No.: 15/176,039

(22) Filed: Jun. 7, 2016

(51) Int. Cl. A61F 5/449; A61F 5/443

(52) U.S. Cl. CPC A61F 5/449; A61F 5/443

(57) ABSTRACT

A support device for a colostomy pouch has an elongated body having two ends, comprising a closable main pocket having an outward-facing front and a rear, an opening in the rear of the pocket sized to accommodate a colostomy pouch, a strap attached to each end, a fastener at the end of one or both straps. In an embodiment, one or more accessories pockets are affixed on the bag body. The opening may also have an expansion slit at the side of the opening, having a closure, wherein when the closure is released the slit opens and causes a larger opening. The closure may be a button closure, a hook and loop closure or a snap fit closure. The device may have a lower opening in the bottom of the main pocket to permit egress of the pouch opening for emptying the pouch while wearing the device.

### BACKGROUND OF THE INVENTION

### 1. Field of Invention

The present invention relates to the field of support devices for colostomy bags and devices.

### 2. Description of Related Art

An ostomy pouching system or colostomy bag is a prosthetic medical device that provides a means for the collection of waste from a surgically diverted biological system, for example colon, ileum, or bladder, and the creation of a stoma. Pouching systems are most commonly associated with colostomies, ileostomies, and urostomies.

Pouching systems usually consist of a collection pouch plastic bag, known as a one-piece system or, in some instances involves a mounting plate, commonly called a flange, wafer or a baseplate, and a collection pouch that is attached mechanically or with an adhesive in an airtight seal, known as a two-piece system. The selection of systems varies greatly between individuals and is often based on personal preference and lifestyle. Ostomy pouching systems collect waste that is output from a stoma. The pouching system allows the stoma to drain into a sealed collection pouch, while protecting the surrounding skin from contaminations.

The adhesive adheres strongly to the abdomen, to prevent leaking and allows the user to maintain an active lifestyle. The baseplate may last one or more days before being replaced, being highly dependent on lifestyle, ostomy type and anatomy. Most wafers/baseplates are manufactured using pectin or similar organic material and are available in a wide variety of sizes to accommodate a person's particular anatomy. The internal opening must be the correct size to accommodate the individual's stoma while protecting the skin from contact with waste. The methods for sizing this opening vary depending on the type of wafer/baseplate; some pre-cut sizes are available, some users customize the opening using scissors. Manufacturers have recently introduced moldable wafers than can be shaped by hand without the need for scissors.

Pouches can be broken down into two basic types: open-end (drainable) and closed-end (disposable). Open-end pouches have a resealable end that can be opened to drain the contents of the pouch into a toilet. The end is sealed with either a Velcro-type closure or a simple clip. Closed-end pouches can be removed and replaced with a new pouch once the bag is full or the pouch can be emptied and rinsed. The flange or wafer does not need to be replaced.

The use of open-end vs. closed-end pouches is dependent on the frequency in which an individual needs to empty the contents. Gas is created during digestion, and an airtight pouch will collect this and inflate. To prevent this some pouches are available with special charcoal filtered vents that will allow the gas to escape, and prevent ballooning at night. Some odor can be expelled through the charcoal filter especially if sufficient deodorant is not used in the pouch.

Pouch covers are helpful to disguise the plastic pouch when it is exposed when reaching or other physical activity. These are usually made of cloth and can be decorative or plain to blend in with clothing. Various sources stock sizes for most manufacturers pouches. There are flexible elastic pouch belts available for extreme physical activity but some of these require the pouch to be worn sideways so it does not fill properly and the tight fit causes pancaking of the effluent.

Based on the foregoing, there is a need in the art for a colostomy pouch bag to contain and support the colostomy bag as it fills, and hide its appearance to allow the user to maintain a normal life without being self-conscious.

### SUMMARY OF THE INVENTION

A support device for a colostomy pouch has an elongated body having two ends, comprising a closable main pocket having an outward-facing front and a rear, an opening in the rear of the pocket sized to accommodate a colostomy pouch, a strap attached to each end, a fastener at the end of one or both straps. In an embodiment, one or more accessories pockets are affixed on the bag body.

The accessories pockets may be positioned at each end between the bag body and the straps, and may be adjacent to the main pocket. In an embodiment the main pocket is closable by a zipper, and the zipper may be water resistant.

The fastener may be a squeeze clip. The opening may be reinforced with a leather grommet, or with a rubber grommet. The floor of the main pocket may also be reinforced. The pouch body may be positioned within the main pocket, and the pouch baseplate is external to the body.

The opening may also have an expansion slit at the side of the opening, having a closure, wherein when the closure is released the slit opens and causes a larger opening. The closure may be a button closure, a hook and loop closure or a snap fit closure. The slit is may be reinforced with extra material. The main pocket may be water resistant, and thee pouch comprises a resealable opening, and the device further comprises a lower opening in the bottom of the main pocket to permit egress of the pouch opening for emptying the pouch while wearing the device.

A method of applying a support device over a colospomy pouch has the steps of adhering the pouch to the side of a user's abdomen over a stoma, pushing the bag through an opening in the rear of a support device so that the bag is positioned within a main pocket, passing straps around a user's body and fastening the straps around the body, and closing the main pocket.

It may include the further steps of opening an expansion slit adjacent to the opening to expand the opening, and removing the support device from the colostomy pouch by passing the pouch through the expanded opening.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the colostomy pouch support bag, according to an embodiment of the present invention;

FIGS. 2a, 2b and 2c are views of the colostomy pouch support bag attached over a colostomy pouch to a human body, according to an embodiment of the present invention.

### DETAILED DESCRIPTION OF PREFERRED EMBODIMENTS

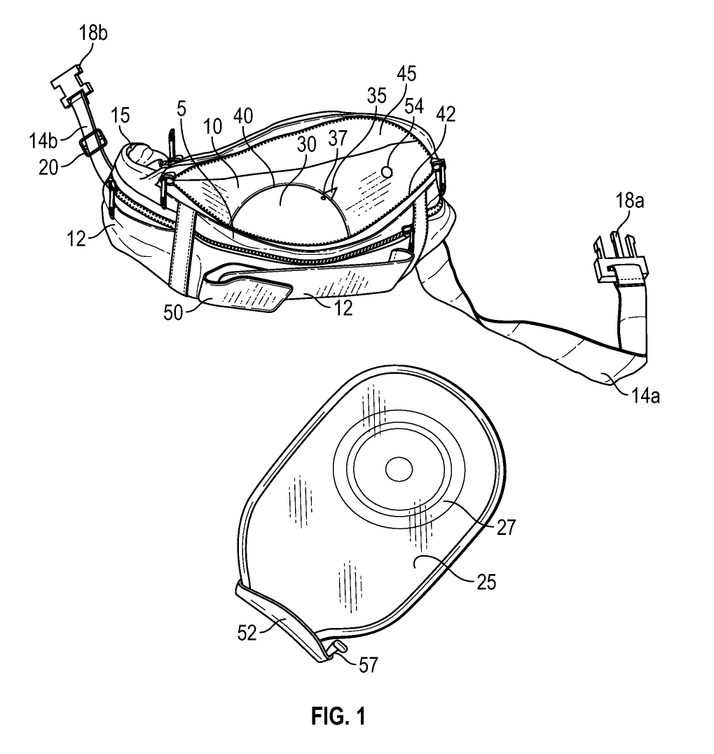

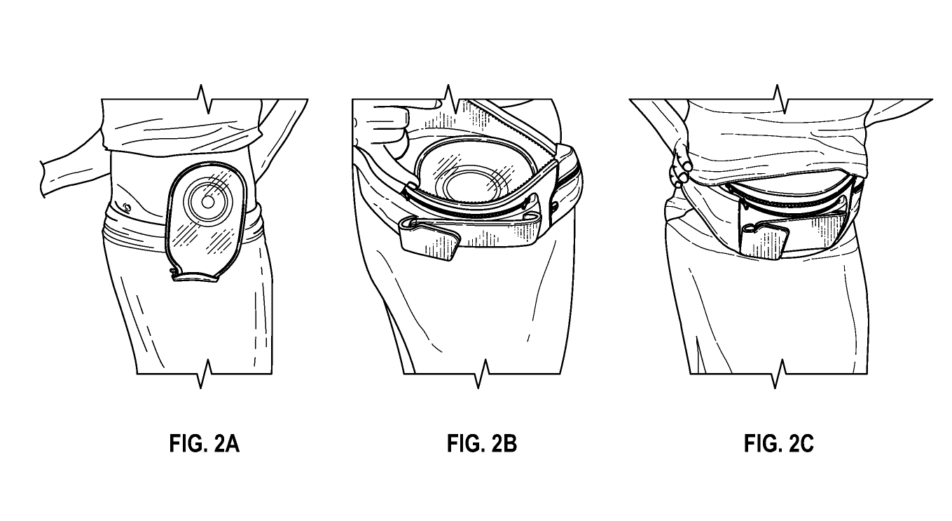

Preferred embodiments of the present invention and their advantages may be understood by referring to FIGS. 1-2c, wherein like reference numerals refer to like elements.

With reference to FIG. 1, the colostomy pouch support bag 2 comprises a support bag body 5 having a single main pocket 10, and one or more accessories pockets 12. The bag body tapers at each end 15. Each end is attached to one or more straps 14a, 14b, which straps extend from the end and terminate in a fastener, typically having a male fastener end 18a and a female fastener end 18b. The fastener may be a squeeze clip or other means for releasably fastening straps, in which case one end will be the female clip 18b and the other end a male clip 18a. One or both straps 14a, 14b may be adjustable for length, by the attachment through the squeeze clip, or by slides 20 along the strap's length. In one embodiment, the first strap 14a has holes and the second strap 14b a buckle for engaging the holes to produce the correct size for the wearer. In one embodiment, the bag has no straps and is held on by the pouch 25 and the adhesive on the baseplate 27 retaining the pouch against the abdomen. As the opening 30 for the pouch is smaller than the pouch, the bag is retained on the pouch, disguising the pouch.

At the rear of the main pocket is an opening 30 that permits the placement of the colostomy pouch within the main pocket 10, while the colostomy pouch baseplate 27 protrudes externally, through the opening 30, to the main pocket 10. The opening diameter is smaller than the colostomy pouch 25 diameter when partially filled, and is also smaller than the baseplate 27. Typical diameter of the opening may be 1½″ to 3½″. This is so the colostomy pouch 25 remains within the support bag 2 despite the user's movements. The opening 30 may have an expansion slit 35 held by a button 37 release on its circumference to open the opening wider to permit the egress of a full colostomy pouch 25 from the main pocket 10, without removing said colostomy pouch 25 from the abdomen. In order to release, the button 37, positioned at the edge of the opening, is opened to reveal a slit that may be expanded to increase the circumference of the opening to permit a full colostomy pouch, to pass through, for example.

The opening is reinforced by rubber or leather grommet 40 sewn or fitted around the opening to prevent wear as the bags are inserted and removed, and to prevent tearing of the opening edge as the bag is manipulated. Additionally, the grommet 40 is smooth to prevent tears in the colostomy pouch. The opening reinforcement grommet 40 may extend in material to cover the bottom of the main pocket to protect the bottom of the pocket from wear and tear.

In an embodiment, the main pocket 10 does not taper but maintains a rectangular shape, such that the zipper 42 may be opened to release three sides of the top flap 45 such that the top flap 45 may be raised without separating. In an embodiment, the main pocket 10 has a sealable bottom opening 50 at the bottom of the front, to permit the egress of the open, resealable end 52 of the pouch for emptying without removal from the support bag 2. The resealable end 52 may then be reclosed and pushed back into the main pocket 10.

The main pocket 10 may have one or more accessories pockets 12 for holding personal effects or further replacement colostomy pouches 25. The pockets 12 may be attached to the body of the bag or may be placed on body extensions at either side, forming connections with the straps 14a, 14b at either end.

In an embodiment, the main pocket 10 is sealed from the interior, to provide a water-resistant contained for the colostomy pouch 25, such that if the pouch 25 leaks the leak is contained within the support bag body 5. In an embodiment, the main pocket 10 is closed by a water-resistant zipper 42, further to prevent leaking, depending on the user's activity. A charcoal vent 54 may be present from the main pocket 10 to the outside to permit venting of the main pocket 10, particularly where the pouch 25 contains a vent to prevent pressure build up.

With reference to FIG. 2a, the user removes the adhesive cover (usually paper), positions the baseplate 27 of the colostomy pouch over the stoma, and pushes the colostomy pouch 25 onto the abdomen around the stoma to adhere the pouch to the abdomen. The adhesive is a removable adhesive that prevents harm to the abdominal skin, and is optimized in regards to absorption, tack and adhesion, flexibility, erosion resistance and ease of removal. The colostomy pouch is preferably open-ended and oriented with the resealable end 52 oriented toward the bottom as recommended in use. The resealable end 52 of the pouch 25 may be closed by hook and loop fasteners or by a clip 57 which bends the bag tightly and maintains the bend to produce a watertight seal.

With reference to FIG. 2b, the user positions the bag over the colostomy pouch, and pushes the empty pouch through the rear opening of the colostomy bag, so the colostomy pouch is contained within the support bag body, wherein the baseplate is positioned on the outside of the opening to adhere to an abdomen. As the colostomy pouch fills, the pouch rests on the bottom of the support bag body such that the weight is supported by, and transferred to, the support bag. The rear opening diameter is smaller than the colostomy pouch diameter when partially filled, and is also smaller than the baseplate. This is so the colostomy pouch remains within the support bag despite the user's movements. The baseplate remains adhered to the abdomen. The straps of the support bag are then connected around the user's waist to retain the support bag on the user's waist using fastening means, and the straps are adjustable to retain the support bag comfortably on the user's waist and hold the weight of a full colostomy bag within the support bag.

With reference to 2c, the user inspects the positioning of the colostomy pouch within the body of the support bag, and closes the body of the bag. The user opens adjacent pockets on the support bag to hold his or her person effects (wallet, phone, etc.) The body of the support bag has an external pocket for replacement colostomy pouches wherein which a user may store other

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A support device for a colostomy pouch, comprising:
a. an elongated body having two ends, comprising:
i. a closable main pocket having an outward-facing front and a rear;
ii. an opening in the rear of the pocket sized to accommodate a colostomy pouch;
b. a strap attached to each end; and
c. a fastener at the end of one or both straps.

2. The support device of claim 1, further comprising one or more accessories pockets on the bag body.

3. The support device of claim 2, wherein the accessories pockets are positioned at each end between the bag body and the straps.

4. The support device of claim 2, wherein the accessories pockets are adjacent to the main pocket.

5. The support device of claim 1, wherein the main pocket is closable by a zipper.

6. The support device of claim 5, wherein the zipper is water resistant.

7. The support device of claim 1, wherein the fastener is a press.

8. The support device of claim 1, wherein the opening is reinforced with a leather grommet.

9. The support device of claim 1, wherein the opening is reinforced with a rubber grommet.

10. The support device of claim 1, wherein the floor of the main pocket is reinforced.

11. The support device of claim 1, further comprising a colostomy pouch, wherein the pouch body is positioned within the main pocket, and the pouch baseplate is external to the body.

12. The support device of claim 1, further comprising an expansion slit at the side of the opening, having a closure, wherein when the closure is released the slit opens and causes a larger opening.

13. The support device of claim 12, wherein the closure is a button closure.

14. The support device of claim 12, wherein the closure is a hook and loop closure.

15. The support device of claim 1, wherein the closure is a snap-fit closure.

16. The support device of claim 12, wherein the slit is reinforced with extra material.

17. The support device of claim 1, wherein the main pocket is water resistant.

18. The support device of claim 1, wherein the pouch comprises a resealable opening, and the device further comprises a lower opening in the bottom of the main pocket to permit egress of the pouch opening for emptying the pouch while wearing the device.

19. A method of applying a support device over a colostomy pouch comprising:
a. adhering the pouch to the side of a user's abdomen over a stoma;
b. pushing the bag through an opening in the rear of a support device so that the bag is positioned within a main pocket;
c. passing straps around a user's body and fastening the straps around the body;
d. and closing the main pocket.

20. The method of claim 19, further comprising the steps of:
a. opening an expansion slit adjacent to the opening to expand the opening; and
b. removing the support device from the colostomy pouch by passing the pouch through the expanded opening.
